(19) United States
(12) Patent Application Publication
(10) Pub. No.: US 2002/0056655 A1
(43) Pub. Date: May 16, 2002

(54) Package for absorbent articles

(75) Inventors: Jeffery R. Cottingham, Appleton, WI (US); James D. McManus, Appleton, WI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Kimberly-Clark Worldwide, Inc.

(21) Appl. No.: 09/965,842

(22) Filed: Sep. 28, 2001

Related U.S. Application Data
(63) Continuation of application No. 09/713,565, filed on Nov. 15, 2000.

(51) Int. Cl. A61L 15/00; A61B 19/02

(52) U.S. Cl. 206/440

(57) ABSTRACT

A package including a preselected plural number of absorbent articles arranged in a stack and packaging. The packaging includes a flexible pocket having a recloseable opening. The pocket at least partially defines a hollow interior having a volume, width and length which are functions of the number of articles, and an article width, length and thickness. The opening has a width and area which are functions of the number of articles, and an article width and thickness.

### CROSS-REFERENCE TO RELATED APPLICATION

This is a continuation of U.S. patent application Ser. No. 09/713,565, filed Nov. 15, 2000, entitled, “PACKAGE FOR ABSORBENT ARTICLES”, which is hereby incorporated by reference in its entirety.

### BACKGROUND OF THE INVENTION

The present invention relates generally to a package, and more particularly to packaging sized as a function of the number and dimensions of the articles held by the packaging.

Absorbent articles such as panty liners, feminine napkins and tampons are frequently carried about in purses, backpacks and briefcases until needed. Typically, the articles are put in these containers loose. Unfortunately, these containers do not always provide a hygienic environment for the articles, and thus the articles can become dirty and/or damaged. Further, the articles can become scattered about in the containers so they are difficult to find when needed.

In the past, specifically designed pouches have been distributed for holding several absorbent articles at a time. These pouches reduce contact between the articles and potentially non-hygienic environments, and make the articles easier to find when needed. The pouches are made from durable materials such as heavy vinyl so they can be reused, but reuse necessitates the pouches be refilled from time to time. Further, the pouches frequently become non-hygienic after extended use, requiring them to be cleaned or discarded and replaced.

To avoid these problems, some persons use clear plastic sandwich bags to hold the articles. These bags usually maintain a hygienic environment for the articles and make the articles easier to find when needed. Although the bags must be filled from time to time, they are readily disposable and replaceable thereby reducing some of the concerns and inconveniences caused by extended use. However, because the bags are transparent, they permit the contents of the bags to be viewed. Some users desire more discreet packaging. Thus, there is a need for a discreet, disposable and economical recloseable package for holding several articles at a time.

One type of packaging which meets the previously mentioned need is described in a co-assigned U.S. patent application, entitled “Package for Absorbent Articles”, filed Nov. 15, 2000, and identified as KCC 4728, which is hereby incorporated by reference in its entirety. This packaging includes a pocket for holding the articles and a opening for withdrawing articles from the pocket when needed. It has been noted that if the pocket or opening is too small, the articles are difficult to withdraw from the packaging. However, if the pocket is too big, the packaging can be unattractive and bulky and the articles can become jumbled in the pocket. Further, if the opening is too big, the articles can fall out of the pocket too easily. Thus, there is a need for packaging having an appropriately sized pocket and opening.

### SUMMARY OF THE INVENTION

Briefly, apparatus of this invention is a package comprising a preselected plural number of absorbent articles arranged in a stack. Each of the absorbent articles has a generally uniform width, length and thickness relative to the other articles. The package further comprises packaging including a flexible pocket for receiving the absorbent articles having a recloseable opening sized and shaped for permitting at least one of the articles to be withdrawn from the pocket. The pocket at least partially defines a hollow interior pocket having a volume when receiving the preselected number of articles less than about twice the number of articles times a product of the article width, length and thickness, and more than about one times the number of articles times the product of the article width, length and thickness.

In another aspect, the package comprises packaging including a flexible pocket for receiving absorbent articles having an opening sized and shaped for permitting at least one of the articles to be withdrawn from the pocket, a flap attached to the pocket adapted for covering the opening, and an adhesive closure for releasably holding the flap in the closed position. Further, the flap and pocket define a hollow interior having a volume when receiving the preselected number of articles less than about twice the number of articles times a product of the article width, length and thickness.

In still another aspect of the present invention, the pocket at least partially defines a hollow interior having a width when the articles are removed therefrom less than about two times the article width and a width when receiving the preselected number of articles more than about one times the article width.

In yet another aspect, the flap and pocket define a hollow interior having a width when receiving the preselected number of articles less than about two times the article width.

In another aspect, the pocket at least partially defines a hollow interior having a length when the articles are removed therefrom less than about two times the article length and a length when receiving the preselected number of articles more than about one times the article length.

In still another aspect, the flap and pocket define a hollow interior having a length when the articles are removed therefrom less than about two times the article length.

In yet another aspect, the opening has a width when the pocket receives the articles less than about two times the article width and more than about one times the article width.

In a final aspect, the opening has an area when the pocket receives the preselected number of articles less than about twice the number of articles times a product of the article width and thickness, and more than about one times the number of articles times the product of the article width and thickness.

Other features of the present invention will be in part apparent and in part pointed out hereinafter.

### BRIEF DESCRIPTION OF THE DRAWINGS

FIG. 1 is a front elevation of a prior art panty liner surrounded by a removable wrapper;

FIG. 2 is a front elevation of a utility package of the present invention showing a flap of the package in a closed position;

FIG. 3 is a front elevation of the utility package showing the flap in an open position;

FIG. 4 is a top plan of the utility package showing the flap in the open position;

FIG. 5 is a front elevation of empty packaging of the utility package;

FIG. 6 is a front elevation of a master package of a first embodiment of the present invention with a portion broken away to show utility packages therein;

FIG. 7 is a front elevation of a master package of a second embodiment of the present invention with a portion broken away to show utility packages therein;

FIG. 8 is a front elevation of a utility package of a second embodiment of the present invention; and

FIG. 9 is a front elevation of a utility package of a third embodiment of the present invention.

Corresponding reference characters indicate corresponding parts throughout the several views of the drawings.

### DETAILED DESCRIPTION OF THE PREFERRED EMBODIMENT

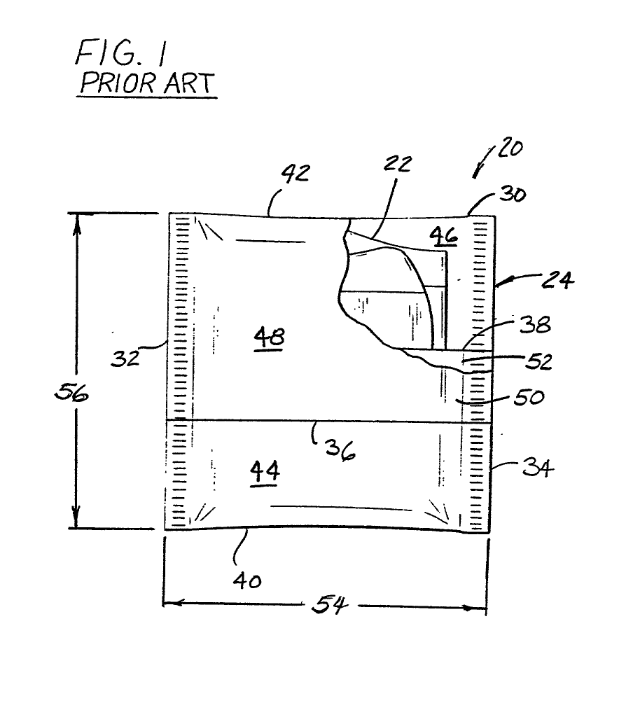

Referring now to the drawings and in particular to FIG. 1, a conventional individually wrapped absorbent article is designated in its entirety by the reference numeral 20. Although the absorbent article 20 shown in FIG. 1 is a panty liner 22 surrounded by a wrapper, generally designated by 24, those skilled in the art will appreciate that the present invention may be applied to other absorbent articles 20 such as feminine napkins, tampons, interlabial pads, other feminine care products, adult care products, child care products and infant care products. Further, those skilled in the art will appreciate that although the illustrated articles 20 are individually wrapped, the articles may be unwrapped without departing from the scope of the present invention.

The panty liner 22 shown in FIG. 1 is folded twice in a conventional manner to present a generally flat and generally rectangular article. Although the folded panty liner 22 may have other sizes without departing from the scope of the present invention, in one embodiment the folded liner has a width of about 50 millimeters, a length of about 70 millimeters and a thickness of about 5 millimeters. It is also envisioned that the panty liner 22 may be unfolded and/or non-rectangular without departing from the scope of the present invention.

Although the wrapper 24 may be made in other ways without departing from the scope of the present invention, in one embodiment the wrapper includes a rectangular sheet 30 having opposing side edges 32, 34 and opposite end edges 36, 38. A first fold 40 in the sheet 30 extending between the side edges 32, 34 forms a bottom of the wrapper 24, and a second fold 42 in the sheet extending between the side edges generally parallel to and above the first fold forms a top of the wrapper. The first fold 40 separates a middle portion 44 of the wrapper 24 from a back portion 46, and the second fold 42 separates the middle portion from a front portion 48 of the wrapper. The side edges 32, 34 of the middle portion 44 are joined to the back portion 46, and the side edges of the forward portion 48 are joined to the middle portion to form opposing sides of the wrapper. A margin 50 of the sheet 30 adjacent the end edge 36 overlaps a margin 52 of the sheet adjacent the end edge 38. It is envisioned that it may be desirable to join the side edges 32, 34 of the forward portion 48 to the back portion 46.

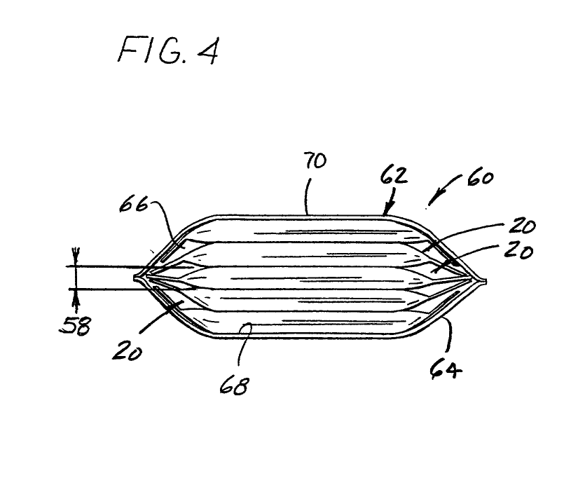

Although the side edges 32, 34 of the rectangular sheet 30 may be joined in other ways (such as with adhesives or by heat sealing) without departing from the scope of the present invention, in one embodiment the side edge margins are joined by conventional mechanical fastening means as shown. Although the wrapped article 20 may have other sizes without departing from the scope of the present invention, in one embodiment the article has a width 54 of about 75 millimeters, a length 56 of about 75 millimeters and a thickness 58 (FIG. 4) of about 5 millimeters. Further, although the dimensions of the article 20 may vary from article to article without departing from the scope of the present invention, in one embodiment the dimensions are generally uniform. In addition, the dimensions may vary within a given article or they may be invariant without departing from the scope of the present invention. Although the wrapper 24 may be made of other materials without departing from the scope of the present invention, in one embodiment the wrapper is made from low density polyethylene sheet material having a thickness of about 38 microns. It is further envisioned that the wrapper 24 may have an adhesive or other closure (not shown) without departing from the scope of the present invention.

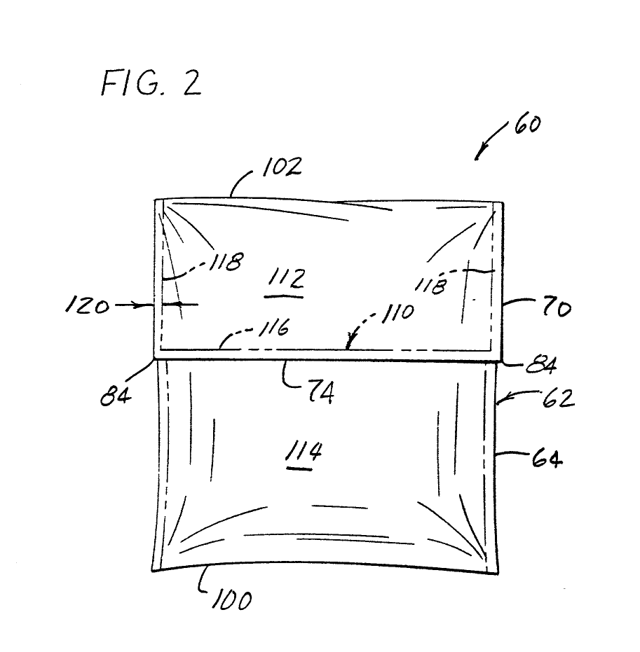

As illustrated in FIGS. 2-4, a utility package of the present invention is designated in its entirety by the reference numeral 60. The package 60 generally comprises a plurality of absorbent articles 20 (FIG. 4) and recloseable packaging, generally designated by 62. As previously mentioned, it is envisioned that the articles 20 may be folded or unfolded and individually wrapped or unwrapped without departing from the scope of the present invention. Although the articles 20 may be arranged in other ways without departing from the scope of the present invention, in one embodiment the articles are arranged in face-to-face relation in a stack as illustrated in FIG. 4. The package 60 includes a number of articles 20 which is selected for the convenience of an end user. For example, the package 60 may include less than about eight absorbent articles 20 to provide a supply of articles for about one week or less. It is envisioned that it may be desirable that the package 60 include about five absorbent articles 20 to provide a supply of articles for one conventional work week (i.e., five days).

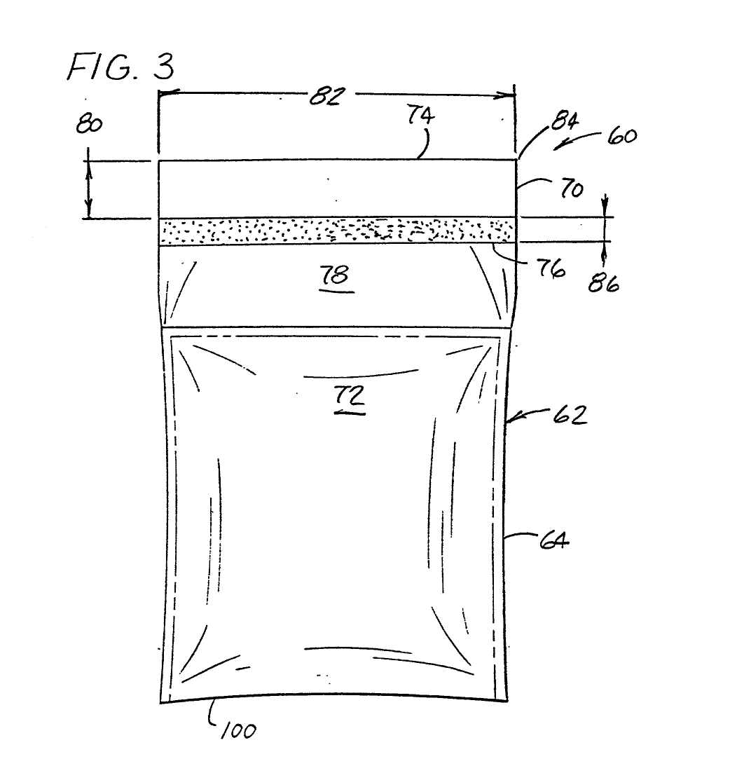

The packaging 62 includes a flexible pocket 64. The pocket 64 has a hollow interior 66 sized and shaped for receiving the preselected number of absorbent articles 20 as shown in FIG. 4. An opening 68 extends into the hollow interior 66 of the pocket 64. The opening 68 is sized and shaped for permitting at least one of the plurality of articles 20 to be withdrawn from the hollow interior 66 of the pocket 64. Further, the packaging 62 includes a flap 70 attached to the pocket 64 adapted for covering the opening 68 to retain the plurality of articles 20 in the hollow interior 66 of the pocket 64. The flap 70 is selectably moveable between an open position as illustrated in FIG. 3 in which the opening 68 is generally unobstructed by the flap to permit at least one article 20 to be withdrawn through the opening and a closed position as shown in FIG. 2 in which the flap covers the opening and an exterior area 72 (FIG. 3) of the pocket 64 to retain the articles in the pocket and to prevent the articles from passing through the opening. In one embodiment, the flap 70 is substantially rectangular and has a substantially straight distal edge 74. However, those skilled in the art will appreciate that the flap 70 may have other shapes such as semi-circular or triangular without departing from the scope of the present invention.

As illustrated in FIG. 3, a closure 76 is positioned on an inner face 78 of the flap 70 for releasably holding the flap in the closed position. It is envisioned that the closure 76 may be positioned on the exterior area 72 of the pocket 64 instead of or in addition to being positioned on the flap 70. Preferably, the closure 76 permits the flap 70 to be positioned in a different location on the pocket 64 during successive closings when successive articles 20 are removed from the interior 66 of the pocket to permit the packaging 62 to conform to the remaining articles in the interior of the pocket. Although other closures 76 may be used without departing from the scope of the present invention, in one embodiment the closure is an adhesive material such as a conventional resealable hot melt adhesive or a resealable two sided tape for releasably holding the flap 70 in the closed position.

Although the closure 76 may be positioned at other locations without departing from the scope of the present invention, in one embodiment the closure is spaced from the distal edge 74 of the flap 70 to permit a free portion of the flap to be grasped and separated from the pocket 64 to grip the flap for releasing the closure. It is envisioned that it may be desirable that the closure 76 be spaced from the distal edge 74 of the flap 70 by a distance 80 of at least about 4 millimeters. It is further envisioned that it may be desirable that the distance 80 be about 16 millimeters.

Although the closure 76 may have other shapes without departing from the scope of the present invention, in one embodiment the closure is an elongate strip of adhesive material extending substantially parallel to the distal edge 74 of the packaging flap 70. In one embodiment, the strip of adhesive material is substantially continuous and uninterrupted along its entire length, but it is envisioned that other embodiments may have adhesive material which is discontinuous and/or interrupted along its length without departing from the scope of the present invention. In addition, it is envisioned that it may be desirable that the strip extend over substantially an entire width 82 of the flap 70 so the corners 84 of the flap are held in place near the pocket 64. Although the elongate strip may have other widths without departing from the scope of the present invention, in one embodiment the strip has a width 86 of less than about 20 millimeters and more than about 2 millimeters. It may be desirable that the width 86 of the strip be about 7 millimeters.

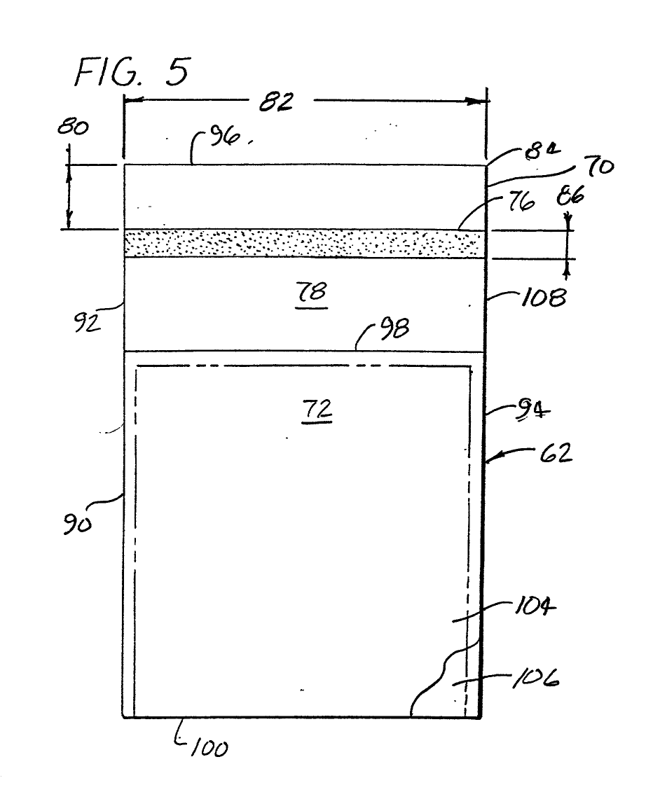

Although the packaging 62 may be made in other ways without departing from the scope of the present invention, in one embodiment illustrated in FIG. 5 the packaging comprises a rectangular sheet 90 having opposing side edges 92, 94 and opposite end edges 96, 98. A first fold 100 extending between the side edges 92, 94 forms a bottom of the packaging 62, and a second fold 102 (FIG. 2) extending between the side edges generally parallel to and above the first fold forms a top of the packaging. The first fold 100 separates a middle portion 104 of the packaging 62 from a back portion 106. The middle and back portions 104, 106, respectively, form the pocket 64 of the packaging 62. The second fold 102 separates the middle portion 104 from a front portion 108 of the packaging 62. The front portion 108 of the packaging 62 forms the flap 70. Although the second fold 102 may be positioned in other locations without departing from the scope of the present invention, in one embodiment the second fold is positioned immediately adjacent the opening 68 and immediately adjacent an upper end of the front portion 108 (i.e., adjacent the edge 98 of the sheet 90). The middle portion 104 is joined with the corresponding back portion 106 along side edges 92, 94 to form opposing sides of the packaging 62. Although the middle and back portions 104, 106 may be joined in other ways (such as with adhesives or by mechanical fastening) without departing from the scope of the present invention, in one embodiment they are joined by conventional heat sealing. Although the packaging 62 may be made from other materials without departing from the scope of the present invention, in one embodiment the packaging is made from a heat sealable polymer sheet material such as a material containing about 80% polyethylene and about 20% other polyolefins having a thickness of about 45 microns available from Shanghai Zihua Enterprise Company, Limited of Shanghai, China. It is further envisioned that the packaging 62 may be made from coated paper, woven material, non-woven material, polyethylene, polypropylene, co-polymers, extruded polymer, thermo-formed materials, and/or cardboard without departing from the scope of the present invention. Although in one embodiment the pocket 64 is substantially free of gussets, in an alternate embodiment the sides of the pocket may include conventional gussets (not shown) to provide the packaging 62 with a substantially flat bottom.

Although the interior 66 of the packaging 62 defined by the flap 70 and the pocket 64 may have other sizes without departing from the scope of the present invention, it may be desirable that the interior have a volume when receiving the preselected number of articles 20 less than about twice the number of articles times a product of the article width 54, length 56 and thickness 58, and more than about one times the number of articles times the product of the article width, length and thickness. As previously mentioned, the number of articles 20 is selected for the convenience of an end user. For example, it is envisioned that it may be desirable that the package 60 include about five absorbent articles 20 to provide a supply of articles for one conventional work week (i.e., five days). Moreover, it may be desirable that the interior 66 have a width when the articles are removed and the pocket 64 is flattened as shown in FIG. 5 less than about twice the article width 54, and a width when receiving the preselected number of articles more than about one times the article width. In one embodiment, the width of the hollow interior 66 when the articles 20 are removed is about 1.2 times the article width. Likewise, it may be desirable that the interior 66 have a length when the articles are removed and the pocket 64 is flattened less than about twice the article length 56, and a length when receiving the preselected number of articles more than about one times the article length. In one embodiment, the length of the hollow interior 66 when the articles 20 are removed is about 1.2 times the article length 56. Further, in one embodiment the opening 68 has a width when the pocket 62 receives the articles 20 less than about two times the article width 54 and more than about one times the article width. It may be desirable that the opening width be about 1.2 times the article width 54. In one embodiment, the opening 68 has an area when the pocket receives the preselected number of articles 20 less than about twice the number of articles times a product of the article width 54 and thickness 58, and more than about one times the number of articles times the product of the article width and thickness.

As illustrated in FIG. 2, it may be desirable that the flap 70 and/or the pocket 64 include a visual indicator, generally designated by 110, for distinguishing the edge 74 of the flap from the pocket. The indicator 110 improves visual identification of the edge 74 of the flap 70 when grasping the flap to move it toward its open position. The visual indicator 110 comprises visually contrasting surface treatments on the flap 70 and the pocket 64. Although other visually contrasting surface treatments may be used without departing from the scope of the present invention, in one embodiment the visually contrasting surface treatments include a first color on at least a portion of an exterior surface or outer face 112 of the flap 70 and a second color on at least a portion of an exterior surface 114 of the pocket 64. Further, it may be desirable that one of the colors be a raw material color of the packaging material and the other color be printed on the packaging 62. For example, if the raw material color of the packaging 62 is white, a lower edge margin 116 and opposing side margins 118 of the flap 70 may be substantially free of printing such as background coloration and text, and a central portion of the flap may include printing of a contrasting color (e.g., blue). The margins 116, 118 are delineated by phantom lines in the drawings. Although the edge margins 116, 118 may have other widths without departing from the scope of the present invention, in one embodiment the edge margins have a width 120 less than about 15 millimeters. It may be desirable that the widths 120 of the edge margins be about 4 millimeters. In addition to omitting printing from the edge margins 116, 118 of the flap 70, it may be desirable that the side margins of the pocket 64 (delineated by phantom lines) be substantially free of printing, to prevent printing discoloration when the sides are heat sealed. Further, it may be desirable that the flap 70 and/or the pocket 64 include a tactile indicator (not shown) for distinguishing the edge 74 of the flap from the pocket to improve tactile identification of the edge of the flap. It is envisioned that the tactile indicator could be a textured portion (e.g., knurling) on an exterior surface of the flap 70 and/or the pocket 64.

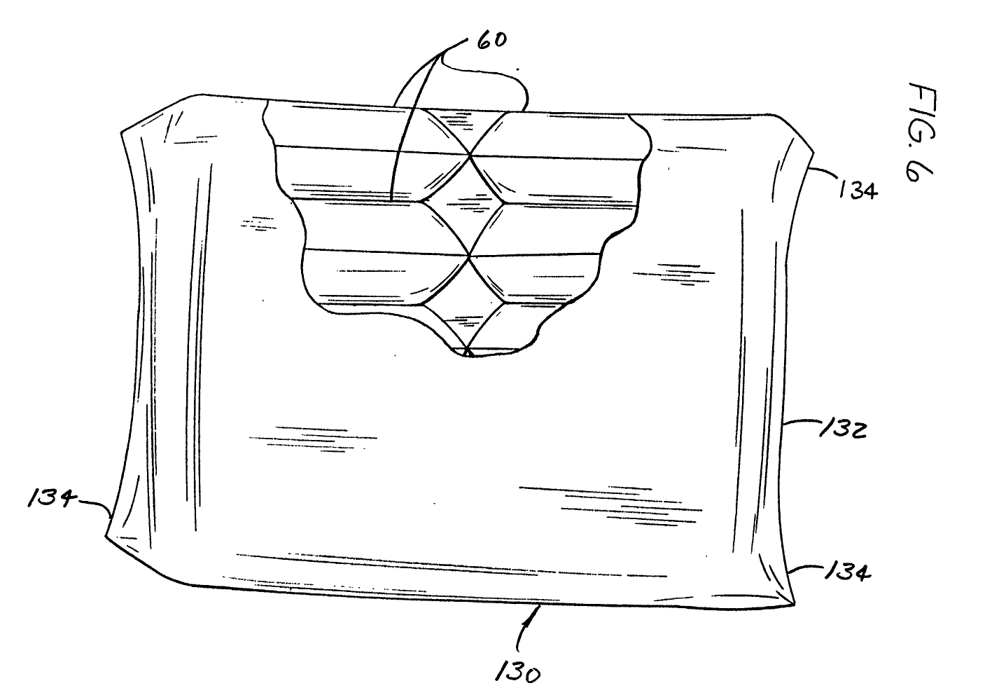

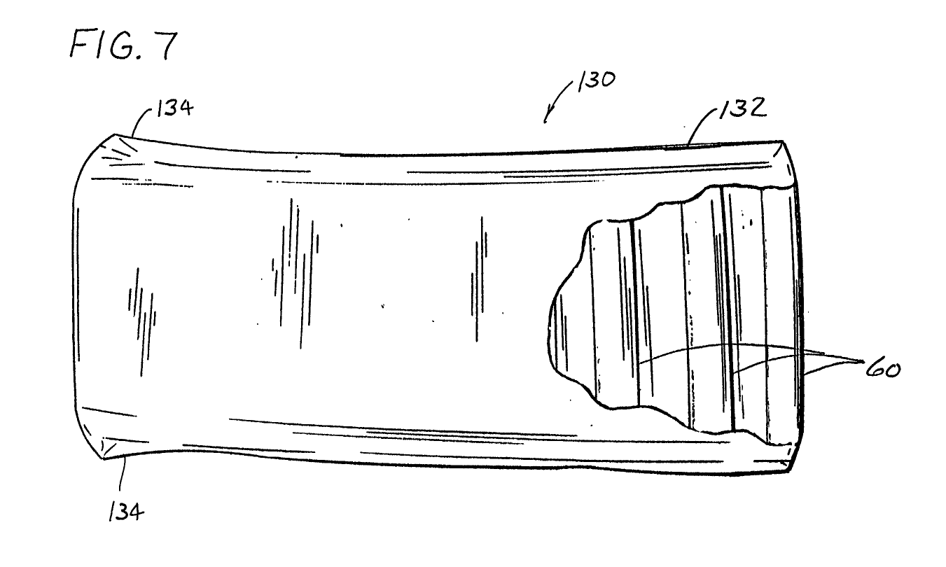

A master package is designated in its entirety by the numeral 130 in FIG. 6. The master package 130 includes a plurality of the utility packages 60 as described above. In addition, the master package 130 includes removable master packaging 132 surrounding the utility packages 60. The master packaging 132 has a hollow interior sized and shaped for receiving the utility packages. It is envisioned that the master packaging may have printing (not shown) on its exterior surface. It is further envisioned that the utility packages 60 may be arranged one or more stacks inside the master packaging 132. For example, the utility packages 60 may be arranged in two stacks as shown in FIG. 6 or arranged in one stack as shown in FIG. 7. Although the master package 130 may include different numbers of utility packages 60 without departing from the scope of the present invention, in the illustrated embodiments the master package includes less than about twelve utility packages. For example, the master package 130 may include about ten utility packages 60 as shown in FIG. 6 or about nine utility packages as shown in FIG. 7. Further, it may be desirable that one or more sides of the master packaging 132 include conventional gussets 134 to provide the packaging with flat sides. Although the master packaging 132 may be made of other materials without departing from the scope of the present invention, in one embodiment the master packaging is made from polyethylene sheet material having a thickness of about 45 microns using conventional packaging methods.

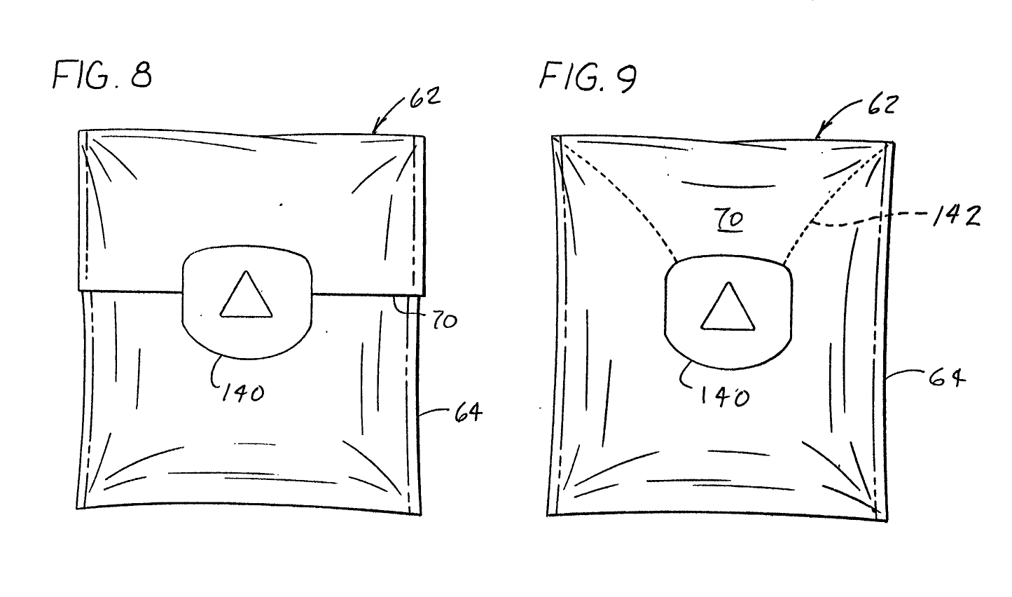

It is envisioned that the utility packages 60 may have alternative constructions without departing from the scope of the present invention. For instance, as illustrated in FIG. 8 a conventional adhesive tab closure 140 may be used to hold the flap 70 in its closed position against the pocket 64 of the packaging 62. Another embodiment shown in FIG. 9 is similar to that of FIG. 8 except that the flap 70 has a generally triangular configuration and is initially joined to the pocket 64 by frangible perforations 142.

In view of the above, it will be seen that the several objects of the invention are achieved and other advantageous results attained.

When introducing elements of the present invention or the preferred embodiment(s) thereof, the articles “all, Ivan”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. A package comprising:
a preselected plural number of absorbent articles arranged in a stack, each of said absorbent articles having a generally uniform width relative to other articles in said number of articles, a generally uniform length relative to the other articles, and a generally uniform thickness relative to the other articles; and
packaging including a flexible pocket for receiving said absorbent articles having a recloseable opening sized and shaped for permitting at least one of said articles to be withdrawn from the pocket, wherein said pocket at least partially defines a hollow interior having a volume when receiving the preselected number of articles less than about twice the number of articles times a product of said article width, length and thickness, and more than about one times the number of articles times said product.

2. A package comprising:
a preselected plural number of absorbent articles arranged in a stack, each of said absorbent articles having a generally uniform width relative to other articles in said number of articles, a generally uniform length relative to the other articles, and a generally uniform thickness relative to the other articles; and
packaging including:
a flexible pocket for receiving said absorbent articles having an opening sized and shaped for permitting at least one of said articles to be withdrawn from the pocket;
a flap attached to the pocket adapted for covering the opening to retain said number of absorbent articles in the pocket, said flap being selectably moveable between an open position in which the opening is generally unobstructed by said flap to permit said at least one article to be withdrawn through the opening and a closed position in which the flap covers the opening and an exterior area of the pocket to retain said articles in the pocket and to prevent said at least one article from passing through the opening; and
an adhesive closure positioned on at least one of said pocket and said flap for releasably holding said flap in the closed position;
wherein the flap and pocket define a hollow interior having a volume when receiving the preselected number of articles less than about twice the number of articles times a product of said article width, length and thickness.

3. A package comprising:
a preselected plural number of absorbent articles arranged in a stack, each of said absorbent articles having a generally uniform width relative to other articles in said number of articles; and
packaging including a flexible pocket for receiving said absorbent articles having a recloseable opening sized and shaped for permitting at least one of said articles to be withdrawn from the pocket, wherein said pocket at least partially defines a hollow interior having a width when the articles are removed therefrom less than about two times said article width and a width when receiving the preselected number of articles more than about one times said article width.

4. A package as set forth in claim 3 wherein the hollow interior width is about 1.2 times said article width when the articles are removed therefrom.

5. A package comprising:
a preselected plural number of absorbent articles arranged in a stack, each of said absorbent articles having a generally uniform width relative to other articles in said number of articles; and
packaging including:
a flexible pocket for receiving said absorbent articles having an opening sized and shaped for permitting at least one of said articles to be withdrawn from the pocket;
a flap attached to the pocket adapted for covering the opening to retain said number of absorbent articles in the pocket, said flap being selectably moveable between an open position in which the opening is generally unobstructed by said flap to permit said at least one article to be withdrawn through the opening and a closed position in which the flap covers the opening and an exterior area of the pocket to retain said articles in the pocket and to prevent said at least one article from passing through the opening; and
an adhesive closure positioned on at least one of said pocket and said flap for releasably holding said flap in the closed position;
wherein the flap and pocket define a hollow interior having a width when receiving the preselected number of articles less than about two times said article width.

6. A package as set forth in claim 5 wherein the hollow interior width is about 1.2 times said article width when the articles are removed therefrom.

7. A package comprising:
a preselected plural number of absorbent articles arranged in a stack, each of said absorbent articles having a generally uniform length relative to other articles in said number of articles; and
packaging including a flexible pocket for receiving said absorbent articles having a recloseable opening sized and shaped for permitting at least one of said articles to be withdrawn from the pocket, wherein said pocket at least partially defines a hollow interior having a length when the articles are removed therefrom less than about two times said article length and a length when receiving the preselected number of articles more than about one times said article length.

8. A package as set forth in claim 7 wherein the hollow interior length is about 1.2 times said article length when the articles are removed therefrom.

9. A package comprising:
a preselected plural number of absorbent articles arranged in a stack, each of said absorbent articles having a generally uniform length relative to other articles in said number of articles; and
packaging including:
a flexible pocket for receiving said absorbent articles having an opening sized and shaped for permitting at least one of said articles to be withdrawn from the pocket;
a flap attached to the pocket adapted for covering the opening to retain said number of absorbent articles in the pocket, said flap being selectably moveable between an open position in which the opening is generally unobstructed by said flap to permit said at least one article to be withdrawn through the opening and a closed position in which the flap covers the opening and an exterior area of the pocket to retain said articles in the pocket and to prevent said at least one article from passing through the opening; and
an adhesive closure positioned on at least one of said pocket and said flap for releasably holding said flap in the closed position;
wherein the flap and pocket define a hollow interior having a length when the articles are removed therefrom less than about two times said article length.

10. A package as set forth in claim 9 wherein the hollow interior length is about 1.2 times said article length when the articles are removed therefrom.

11. A package comprising:
a preselected plural number of absorbent articles arranged in a stack, each of said absorbent articles having a generally uniform width relative to the other articles; and
packaging including a flexible pocket for receiving said absorbent articles having an opening sized and shaped for permitting at least one of said articles to be withdrawn from the pocket, wherein the opening has a width when the pocket receives the articles less than about two times said article width and more than about one times said article width.

12. A package as set forth in claim 11 wherein the opening width is about 1.2 times said article width when the articles are removed therefrom.

13. A package comprising:
a preselected plural number of absorbent articles arranged in a stack, each of said absorbent articles having a generally uniform width relative to other articles in said number of articles, and a generally uniform thickness relative to the other articles; and
packaging including a flexible pocket for receiving said absorbent articles having an opening sized and shaped for permitting at least one of said articles to be withdrawn from the pocket, wherein the opening has an area when the pocket receives the preselected number of articles less than about twice the number of articles times a product of said article width and thickness, and more than about one times the number of articles times said product.
